(19) United States
(12) Patent Application Publication
(10) Pub. No.: US 2024/0024671 A1
(43) Pub. Date: Jan. 25, 2024

(54) BURST STIMULATION PATTERN FOR NEUROMODULATION

(71) Applicants: Universiteit Antwerpen, Antwerpen (BE); Universitair Ziekenhuis Antwerpen, Edegem (BE)

(72) Inventor: Stefan De Wachter, Wilrijk (BE)

(73) Assignees: Universiteit Antwerpen, Antwerpen (BE); Universitair Ziekenhuis Antwerpen, Edegem (BE)

(21) Appl. No.: 18/223,218

(22) Filed: Jul. 18, 2023

Related U.S. Application Data
(60) Provisional application No. 63/391,827, filed on Jul. 25, 2022.

(51) Int. Cl. A61N 1/36

(52) U.S. Cl. CPC A61N 1/36007; A61N 1/36178; A61N 1/36175; A61N 1/36171; A61N 1/36139

(57) ABSTRACT

The application discloses methods of ensuring pelvic health and/or treating a disease or disorder characterized by a dysfunctional autonomous nervous system in a subject with neuromodulation. The methods are characterized by the application of a burst stimulation pattern comprising a plurality of groups of electric pulses of high frequencies to the subject.

### CROSS-REFERENCE TO RELATED APPLICATION

This application claims the benefit of U.S. Provisional Patent Application No. 63/391,827, filed on Jul. 25, 2022, the contents of which are herein incorporated by reference in their entirety into the present application.

### FIELD OF THE INVENTION

The present application relates to methods of ensuring pelvic health and treating a disease or disorder characterized by a dysfunctional autonomic nervous system using neuromodulation and applying a burst stimulation pattern.

### BACKGROUND OF THE INVENTION

Sacral neuromodulation (SNM) is a third-line therapy for patients with an overactive bladder (OAB) or chronic non-obstructive urinary retention (NOUR). SNM success rates for these patients are often defined in literature as an improvement in symptomatology of 50% or more compared to baseline results. Nowadays, these success rates range from approximately 80% after one month to approximately 60% after 5 years of stimulation and approximately 45% of patients become complete symptom free. Nevertheless, still a significant number of patients cannot be treated with the currently available sacral neuromodulation treatment options.

SNM is currently provided in tonic electrical stimulation which can subsequently be adjusted to patient preference within a specific range of parameters, depending on its efficacy. Currently applied conventional SNM protocols are characterized by stimulation patterns wherein electrical pulses are applied at a relative low frequency and not burst pulses are applied. Since the early days of SNM, the standard pulse frequency ranges between 14-16 Hz for both urological and bowl dysfunctions. This standard pulse frequency is generally combined with a pulse width of 210 μs. It has been further shown that frequency increases could lead to significant clinical improvements, although in these studies only frequency increases up to 31 Hz were applied. Assmann, Douven and colleagues further reviewed the clinical effect of stimulation parameters for SNM and showed that only narrow ranges of stimulation parameters with low frequencies were examined in human patients (Assmann et al., Neuromodulation 2020, 23(8): 1082-93). In the accompanying paper in which SNM stimulation parameters in animal studies were reviewed, significant improvement of urinary tract dysfunction has been found in several studies investigating SNM frequencies below 100 Hz. Although not all studies on the effects of SNM frequency were performed with similar stimulation intensity or pulse width, the use of SNM at various frequencies (0.01-100 Hz) in animal models was shown to significantly reduce the number of bladder contractions per minute or to reduce bladder pressure using frequencies from 0.05 Hz to 50 Hz, with the best results applying 4, 7.5 and 10 Hz stimulation (Douven et al., Neuromodulation 2020, 23(8): 1094-107). Furthermore, SNM applied in Burst patterns (4-6 pulse Burst; interburst frequency 0.01-1 Hz, intraburst frequency 0.1-1000 Hz) showed similar results to conventional SNM (con-SNM) in female rats, with an optimal setting of a four-pulse Burst 1 Hz interburst frequency and 40 Hz intraburst frequency (Su et al., Neuromodulation 2017, 20(8): 787-92). In recent years, Burst patterns are hardly applied for lower urinary tract dysfunction.

### SUMMARY

In the present invention, the inventors have developed methods using burst stimulus neuromodulation to treat subjects with a dysfunctional autonomous nervous system, in particular subjects with an overactive bladder or chronic non-obstructive urinary retention. As corroborated in the experimental section, stimulation of sacral nerve roots with specific burst patterns comprising groups of high frequency electrical pulses resulted in a strong bladder response with higher bladder and urethral pressures as compared to the conventional sacral neuromodulation methods known from the prior art.

In a first aspect, a method of ensuring pelvic health and/or treating a disease or disorder characterized by a dysfunctional autonomous nervous system in a subject with neuromodulation is provided. The method comprises applying electric pulses to the subject through a set of one or more electrodes of an electrical lead that is operably linked to a pulse generator, and wherein the method comprises applying a burst pattern comprising a plurality of groups of electric pulses to the subject through a set of one or more electrodes of an electrical lead in the proximity of the sacral plexus and/or the pelvic plexus of the subject.

In some embodiments, the dysfunctional autonomic nervous system is selected from the group consisting of a muscle control disorder of the colon or bladder, a sexual dysfunction, an inflammatory disorder of the bladder and/or intestines, chronic visceral pain, chronic bladder pain and chronic pelvic pain. In some further embodiments, the muscle control disorder of the colon or bladder is a muscle disorder of one or more sphincters of the colon or bladder.

In some embodiments, the method comprises a step of positioning the one or more electrodes of the electrical lead in the proximity of the sacral and/or pelvic plexus of the subject by guiding the one or more electrodes through the third or fourth sacral foramen.

In some embodiments, the intraburst frequency is constant throughout the entire burst pattern.

In some embodiments, the amplitude of the first pulse of each group of electric pulses is the same, wherein the amplitude of a subsequent pulse is higher than the amplitude of its preceding pulse within a group of electric pulses and wherein the amplitude of each subsequent pulse gradually increases compared to the preceding pulse within said group and wherein a passive charge balance occurs during each interburst interval.

In some embodiments, the method further comprises detecting one or more of the bladder pressure (pᵥₑₛ), proximal urethral pressure (pᵤᵣₐ₁) and mid urethral pressure (pᵤᵣₐ₂) of said subject.

In some embodiments, applying the burst pattern to the subject increases the bladder pressure, proximal urethral pressure, the mid urethral pressure, or a combination thereof.

In some embodiments, the method comprises determining the nature of the dysfunction of the autonomous nervous system in the subject and adjusting one or more of the application location, amplitude and interburst frequency of the burst pattern to specifically correct said dysfunction.

These and further aspects and preferred embodiments of the invention are described in detailed in the following sections and in the appended claims. The subject-matter of the appended claims is hereby specifically incorporated in this specification.

### BRIEF DESCRIPTION OF DRAWINGS

FIG. 1A-1C. The effect of conventional sacral neuromodulation (Con-SNM) and Burst SNM with increasing intensities on bladder and urethral pressure. In the con-SNM protocol, stimulation parameters were 210 μs as pulse width and 14 Hz as frequency in combination with incrementally increasing amplitudes (1 mA to 5 mA). For the burst SNM protocol, different interburst frequencies (10-20-40 Hz) were evaluated together with a constant intraburst frequency of 500 Hz and a pulse width of 1000 μs. Incremental stimulation intensities were applied within the burst (increasing amplitudes from 1-2-3-4 mA). Bladder pressure (Δpᵥₑₛ) is presented in A, proximal urethral pressure (Δpᵤᵣₐ₁) in B and mid urethral pressure (Δpᵤᵣₐ₂) in C.

FIG. 2A-2C. Burst SNM paradigms with a similar total charge (200 μC/s). Burst paradigms represented here are Burst 10 Hz (10 Hz interburst frequency with a 4 mA stimulation amplitude), Burst 20 Hz (20 Hz interburst frequency with a 2 mA stimulation amplitude) and Burst 40 Hz (40 Hz interburst frequency with a 1 mA stimulation amplitude). In all burst paradigms the intraburst frequency is 500 Hz. Bladder pressure (Δpᵥₑₛ) is presented in A, proximal urethral pressure (Δpᵤᵣₐ₁) in B and mid urethral pressure (Δpᵤᵣₐ₂) in C.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The inventors of the present application have found that neuromodulation of the autonomous nervous system of the sacral and pelvic region using burst stimulation can be used to ensure pelvic health or to treat a disease or disorder characterized by a dysfunctional autonomous nervous system. More specifically, and as also corroborated in the experimental section, neuromodulation of the sacral plexus and/or the pelvic plexus using a specific burst stimulation pattern significantly increased the bladder pressure resulting in an improvement of the symptoms associated with an overactive bladder or chronic non-obstructive urinary retention, as compared to the conventional sacral neuromodulation patterns without burst stimulation. Sacral neuromodulation has already been used to treat both urinary and faecal storage and evacuation dysfunctions when conservative treatment options are not sufficient. However, the conventional sacral neuromodulation protocols that are known today are characterized by conventional stimulation patterns wherein electrical pulses are applied that have a relative low frequency. More specific, pulse frequencies in these conventional stimulation patterns are generally not higher than 50 Hz, and typically frequencies of 5-20 Hz are applied. These pulse frequencies are then combined with very low interburst frequencies, typically only frequencies between 0.1 and 1 Hz. With the present invention, the inventors show that application in the pelvic-sacral region of a burst stimulation pattern that is characterized by the presence of several bursts or groups of electric pulses of a high frequency, such as a frequency of 500 to 1000 Hz, significantly improved the symptoms in patients with an overactive bladder or chronic non-obstructive urinary retention. In addition, the burst stimulation pattern of the present invention is further characterized in that it shows a relatively high interburst frequency, that typically ranges between 10 and 100 Hz.

A first aspect of the invention thus provides a method of ensuring pelvic health and/or treating a disease or disorder characterized by a dysfunctional autonomic nervous system in a subject with neuromodulation, wherein the method comprises applying a burst pattern comprising a plurality of groups of electric pulses to the subject through a set of one or more electrodes of an electrical lead located in the proximity of the sacral plexus and/or pelvic plexus of the subject, said electrical lead being operably linked to a pulse generator.

The present invention thus provides methods of ensuring pelvic health and/or treating a disease or disorder characterized by a dysfunctional autonomous nervous system in a subject by applying neuromodulation with a burst stimulation pattern that comprises a plurality of groups of electrical pulses of a higher frequency. Typically, the frequency of the electrical pulses in said groups are of a frequency that is above 100 Hz, preferably above 200 Hz, more preferably above 500 Hz. Also typical for the invention is that the burst pattern is applied to the subject through a set of one or more electrodes of an electrical lead located in the proximity of the sacral plexus and/or the pelvic plexus of the subject and wherein the electrical lead is operably linked to a pulse generator.

As used herein, “neuromodulation” refers to the alteration of nerve activity through targeted delivery of a stimulus, such as electrical stimulation, to specific neurological sites in the body. Electric neuromodulation thus refers to electric stimulation of specific neural circuits, such as neuronal circuits located in the brain, the spine, in order to influence the nerve activity. In the methods as taught herein, neuromodulation of the sacral region is envisaged, in particular neuromodulation of the sacral plexus and/or the pelvic plexus.

The “sacral plexus” are used herein refers to a network of nerves or neurons by the lumbosacral trunk (L4, L5 in human) and sacral spinal nerves (S1-S4 in human). The lumbosacral trunk is a thick nervous band that arises from the merger of the anterior/ventral rami of the last two lumbar spinal nerves (L4 and L5). Upon originating, the trunk descends over the wing of the sacrum and joins the sacral spinal nerves to form the sacral plexus. The sacral plexus is located on the posterior pelvic wall, posterior to the internal iliac vessels and ureter and anterior to the piriformis muscle.

The “pelvic plexus” is the network of neurons that govern visceral tissues involved in eliminative and reproductive functions. It is the singular site in the autonomous nervous system where sympathetic and parasympathetic neurons occur in the same ganglia.

The methods of the invention are thus characterized in that one or more electrodes of an electrical lead are located in the proximity of the sacral plexus and/or the pelvic plexus of the subject and that a pulse generator that is operably linked to the electrical lead is used to deliver electrical stimulation energy to the sacral plexus and/or the pelvic plexus of the subject. In some embodiments, the one or more electrodes of the electrical lead are located in the proximity of the sacral plexus of the subject. In some embodiments, the one or more electrodes of the electrical lead are located in the proximity of the pelvic plexus of the subject. In some embodiments, the one or more electrodes of the electrical lead are located in the proximity of both the sacral plexus and the pelvic plexus of the subject. As used herein, the location of the one or more electrodes in the proximity of the sacral and/or pelvic plexus in the subject is to be understood as situated in a close region around the sacral and/or pelvic plexus or in a close region around one or more neurons that are part of the sacral and/or pelvic plexus. The location of the one or more electrodes in the proximity of the sacral and/or pelvic plexus is also to be understood as positioning the one or more electrodes at such a distance from the neurons that they can still affect and stimulate the neurons. It should be understood that the location of the electrodes can differ in each subject and can be influenced by the subject's anatomy. In more specific embodiments, the location of the one or more electrodes in the proximity of the sacral and/or pelvic plexus in the subject is to be understood as the situation wherein the electrodes are located within a distance of 0.10 mm to 50.0 mm of one or more neurons of the sacral and/or pelvic plexus. In some embodiments, the one or more electrodes are located within a distance of 1.0 mm to 20.0 mm of one or more neurons of the sacral and/or pelvic plexus. For example, the one or more electrodes may be located within a distance of about 10.0 mm of one or more neurons of the sacral and/or pelvic plexus.

The one or more electrodes of the electrical lead are thus positioned near the sacral and/or pelvic plexus of the subject so that they are able to deliver electrical stimulation energy to the neurons of the sacral and/or pelvic plexus of the subject. In particular embodiments, the one or more electrodes of the electrical lead are positioned in proximity of one or more of the L4, L5, S1, S2, S3, and S4 nerves of the human subject so that they can deliver electrical energy to these L4, L5, S1, S2, S3, and S4 nerves, preferably the one or more electrodes are positioned so that they are able to stimulate the S3 and/or S4 nerves.

The methods as taught herein may further comprise a step of positioning the one or more electrodes of the electrical lead in the proximity of the sacral and/or pelvic plexus of the subject by guiding the one or more electrodes through a sacral foramen, which is an opening in the sacral vertebrae. In some embodiments, electrodes can be guided through both a posterior and an anterior sacral foramen. In some embodiments, electrodes are guided through either a posterior or anterior sacral foramen. Preferably, the one or more electrodes are positioned in the proximity of the sacral and/or pelvic plexus of the subject by guiding the one or more electrodes through the third or fourth sacral foramen. In some embodiments, electrodes can be guided through two or more sacral foramina, wherein an electrical lead with one or more electrodes is guided through each of the sacral foramen. In some embodiments, electrodes are guided through both the third and fourth sacral foramina, wherein an electrical lead with one or more electrodes is guided through the third and fourth sacral foramina. As a result, the one or more electrodes are positioned in close proximity of the S3 and/or S4 nerves, in particular at a location that allows electrostimulation of the S3 and/or S4 nerves.

The methods as taught herein are typically to ensure pelvic health and/or to treat a disease or disorder characterized by a dysfunctional autonomous nervous system in a subject, preferably in a human subject.

In some embodiments, the methods as taught herein are provided to ensure pelvic health in a subject, preferably a human subject. Pelvic health as used herein is to be understood as the correct functioning and management of the bladder, bowel and reproductive organs. In particular embodiments, the methods as taught herein are to ensure pelvic health in female subjects, preferably to treat and/or prevent disorders associated with the female pelvis, such as disorders of the bladders, parts of the bowel or intestine, all reproductive organs (uterus, ovaries, vagina), and the muscles, ligaments and other tissues that are associated with these organs.

In some embodiments, the methods as taught herein are provided to treat a disease or disorder characterized by a dysfunctional autonomous nervous system in a subject, preferably a human subject. Preferably, the methods are to treat a disease or disorder characterized by a dysfunctional nervous system located in the urogenital region of the subject. In some embodiments, disorders or diseases with a dysfunctional autonomous nervous system is selected from the group consisting of a muscle control of the colon or bladder, a sexual dysfunction, an inflammatory disorder of the bladder or intestines, chronic visceral pain, chronic bladder pain and chronic pelvic pain. In some embodiments, the muscle control disorder of the colon or bladder is a muscle disorder of one or more sphincters of the colon or bladder. In general the sphincters of the colon or bladder are part of the pelvic floor.

In some embodiments, the muscle control disorder is a muscle control disorder of one or both of the urethral sphincters. The sphincters of the bladder are also known as urethral sphincters and are muscles used to control the exit of urine in the urinary bladder through the urethra. In humans, two urethral sphincters are present: an external urethral sphincter located in the deep perineal pouch, at the bladder's distal inferior end in females and inferior to the prostate in males; and an internal urethral sphincter located at the bladder's inferior end and the urethra's proximal end at the junction of the urethra with the urinary bladder.

In some embodiments, the muscle control disorder is a muscle control disorder of the colon sphincter, also known as the anal sphincter. In some embodiments, the muscle control disorder is a disorder of the internal anal sphincter, of the external anal sphincter, or of both the internal and the external anal sphincter. In humans, the external anal sphincter is a flat plane of skeletal muscle fibers, elliptical in shape and intimately adherent to the skin surrounding the margin of the anus. The internal anal sphincter in humans is a ring of smooth muscle that surrounds about 2.5-4.0 cm of the anal canal.

In some embodiments, the muscle control disorder of the colon or bladder is a muscle control disorder of the muscles of the pelvic floor.

In some preferred embodiments, the methods as taught herein are to treat a disease or disorder characterized by a muscle control disorder of the colon or bladder, such as an overactive bladder (OAB) or chronic non-obstructive urinary retention (NOUR).

In some embodiments, the methods as taught herein are to treat a sexual dysfunction disorder, such as an erectile dysfunction.

In some embodiments, the methods as taught herein are to treat an inflammatory disorder of the bladder or the intestines. For example, the methods as taught herein can be used to treat inflammatory bowel disease (IBD).

As used herein, the terms “subject”, “individual” or “patient” are used interchangeably throughout this specification, and typically and preferably denote humans, but may also encompass reference to non-human animals, preferably warm-blooded animals, even more preferably mammals, such as, e.g. non-human primates, rodents, canines, felines, equines, ovines, porcines, and the like. The term “non-human animals” includes all vertebrates, e.g., mammals, such as non-human primates (particularly higher primates), sheep, dog, rodent (e.g., mouse or rat), guinea pig, goat, pig, cat, rabbits, cows, and non-mammals such as chicken, amphibians, reptiles, etc. In certain embodiments, the subject is a mammal. In some embodiments, the subject is a non-human mammal.

In some preferred embodiments of the methods as taught herein, the subject is a human subject. In other embodiments, the subject is an experimental animal or animal substitute as a disease model. In some preferred embodiments, the subject is a male subject or a female subject.

Suitable subjects may include subjects, preferably humans, that are diagnosed with or suspected to have a disease or disorder characterized by a dysfunctional autonomous nervous system. In some embodiments, the subjects are diagnosed with a muscle control disorder of the colon or bladder, a sexual dysfunction, an inflammatory disorder of the bladder or intestines, chronic visceral pain, chronic bladder pain or chronic pelvic pain. In some embodiments, the subjects that are diagnosed with a muscle control disorder of the bladder or colon are subjects that are diagnosed with a muscle control disorder of one or more sphincters of the bladder or colon. In some preferred embodiments, the subjects are diagnosed with overactive bladder or chronic non-obstructive urinary retention.

Suitable subjects may also include subjects that are suffering from an impaired pelvic health, such as any complains or disease symptoms associated with the pelvis.

As used throughout this specification, the terms “therapy” or “treatment” refer to interventions, such as neurostimulating interventions, that result in the alleviation or measurable lessening of one or more symptoms or measurable markers of a pathological condition such as a disease or disorder. The terms encompass primary treatments as well as neo-adjuvant treatments, adjuvant treatments and adjunctive therapies. The terms “therapy” or “treatment” broadly refer to interventions that result in the alleviation or measurable lessening of one or more symptoms or measurable markers of pelvic disorders or disorders associated with a dysfunctional autonomic nervous system. Measurable lessening includes any clinically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments.

The methods as taught herein are thus characterized in that a burst pattern is applied through a set of one or more electrodes of an electrical lead wherein the electrical lead is operably linked to a pulse generator.

In some embodiments, the burst pattern is generated through a set of one or more electrodes of only one electrical lead that is operably linked to a pulse generator. In some embodiments, each of the electrical lead may comprise only one electrode. In some embodiments, the electrical lead may comprise a plurality of electrodes, such as two, three, four or even more than four, electrodes. In some embodiments, the burst pattern is thus applied using two or more electrodes, such as three, four, five, six or even more, of one electrical lead that is operably linked to a pulse generator.

In some embodiments, a burst pattern is applied through a set of one or more electrodes of a plurality of electrical leads that are operably linked to a pulse generator. For example, two electrical leads can be operably linked to the pulse generator. In some embodiments, each of the electrical leads may comprise only one electrode. In some embodiments, each of the electrical leads may comprise a plurality of electrodes, such as two, three, four or even more than four, electrodes. In some embodiments, the burst pattern is thus applied using two or more electrodes, such as three, four, five, six or even more, of a plurality of electrical leads that are operably linked to a pulse generator.

As an example, two electrical leads can be operably linked to a pulse generator, wherein each electrical lead comprises one or more electrodes. For example, two electrical leads are operably linked to a pulse generator wherein each electrical lead comprises only one electrode. Or, two electrical leads are operably linked to a pulse generator wherein each electrical lead comprises two or four electrodes.

As used herein, an “electrical lead” refers to an electrical wire or electrical connection that runs between the pulse generator and the location where the electrical stimulation energy needs to be delivered. The leads are thus operably linked to the pulse generator which generates the electrical pulses. The electrical leads comprise one or more electrodes. Generally, the one or more electrodes are located in the part of the electrical lead that is close to the location where the electrical stimulation energy needs to be delivered. The electrical leads or electrical lead wires are thus operably linked to the pulse generator and they include one or more electrodes that allow the delivery of electrical stimulation energy from the pulse generator via the electrical leads and the one or more electrodes to the preferred location in the body, preferably to a location that is close to the sacral and/or pelvic plexus of the subject.

In some embodiments, the pulse generator may be a unilateral lead pulse generator wherein only one electrical lead is operably linked to the pulse generator. In some other embodiments, the pulse generator may be a bilateral lead pulse generator wherein two electrical leads are operably linked to the pulse generator. In some embodiments, the bilateral lead pulse generator is configured to be operably linked with two electrical leads in such a way that the electrical leads are located in the proximity of the left and right branches of the sacral and/or pelvic plexus.

In general the one or more electrodes, the one or more electrical leads and the pulse generator together form the neuromodulation device, also called the electrical stimulation source. The neuromodulation device is configured for applying electrical stimulation pulses to a predetermined site, such as the proximity of the sacral and/or pelvic plexus. In operation, the one or more electrodes, the one or more electrical leads and the pulse generator are all implanted in the subject. It will be understood that any conventional neuromodulation device known by the skilled person can be used in the methods of the invention. However, conventional neuromodulation devices can be further modified to apply burst stimulation to nerve tissue of a patient by modifying the software instructions stored in the devices. Generally, neuromodulation devices typically include a microprocessor and a pulse generation module. The pulse generation module generates the electrical pulses according to a defined pulse width and pulse amplitude and applies the electrical pulse to defined electrode via the electrical lead. The microprocessor controls the operations of the pulse generation module according to the software instructions stored in the device. Neuromodulation devices to generate burst stimulation patterns are adapted by programming the microprocessor to deliver a number of electrical pulses or spikes that are separated by an appropriate interspike or intraburst interval. Thereafter, the programming of the microprocessor causes the pulse generation module to cease pulse generation operations for an interburst interval. The programming of the microprocessor also causes a repetition of the spike generation and cessation of operations for a predetermined number of times. After the predetermined number of repetitions have been completed, the microprocessor can cause burst stimulation to cease for an amount of time (and resume thereafter). The microprocessor can be programmed to allow the various characteristics of the burst stimulus to be set by a physician to allow the burst stimulus to be optimized for a particular patient or pathology. For example, the pulse amplitude, the intraburst interval, the interburst interval, the number of bursts to be repeated in succession, the amplitude of the electrical pulses, and other characteristics can be controlled using respective parameters accessed by the microprocessor during burst stimulus operations. These parameters can be set to desired values by an external programming device via wireless communication with the implantable neuromodulation device.

The method of the invention is thus characterized by applying a burst stimulation pattern to the nerves of the sacral and/or pelvic plexus.

As used herein, a “burst pattern”, also referred to as a “burst stimulation pattern” refers to a pattern that comprises a plurality of groups of electric pulses alternating with periods of relative quiescence. A burst pattern is thus a pattern that comprises a plurality of bursts or burst stimuli alternating with relative ‘silent’ periods. As used herein, the term “burst” or “burst stimulus” refers to a group of electric pulses, also called “spike pulses”, or to a period in the burst pattern that has a much higher discharge rate than surrounding periods in the pattern. A burst is thus a train of action potentials or electric pulses that occurs during a ‘plateau’ or ‘active phase’, followed by a period of relative quiescence called the ‘silent phase’. The burst or burst stimulus is characterized by action potentials or electric pulses that are of high frequency, such as pulses with a frequency of 400 Hz or higher, whereas the silent phase in the burst pattern is characterized by action potentials of low frequency, such as a frequency below 100 Hz.

As used herein the terms “spike”, “pulse”, “electric spike” or “electric pulse can be used interchangeably and refer to an action potential. Yet further, a “burst spike” refers to a spike that is preceded or followed by another spike within a short time interval. A plurality or a group of burst spikes or electric pulses thus forms the burst or burst stimulus.

As used herein, the term “intraburst frequency” refers to the frequency of the burst pattern within a burst or burst stimulus. The intraburst frequency thus refers to the frequency or the number of the electric pulses or spikes within a burst or burst stimulus. It is also referred to as the frequency of the burst pattern in the ‘active phase’ or ‘plateau phase’. As used herein, the term “interburst frequency” refers to the frequency of the burst pattern in between the bursts or bursts stimuli. It thus refers to the frequency of the burst pattern that is applied outside of the electric or spike pulses. It is also referred to as the frequency of the burst pattern in the ‘silent phase’ or ‘quiescent phase’. Generally, in burst stimulation patterns for neuromodulation, the intraburst frequency is higher than the interburst frequency.

As used herein, the “interburst interval”, also referred to as the “inter-spike interval” refers to the interval or the time in between two electric pulse or spike pulses within a burst. Generally, the interburst interval is about 100 ms but it can be shorter or longer, for example 0.5 milliseconds.

As used herein, the “pulse width”, or also referred to as the “spike width” refers to the width or the time or duration of an electric pulse or electric spike.

As used herein, the “amplitude” of an electric pulse or spike refers to the peak current or peak action potential that is delivered in the electric pulses or spikes. The amplitude is expressed in milliAmpére (mA).

In some embodiments, the methods as taught herein comprise applying a burst pattern wherein the interburst frequency between each burst or group of electric pulses or spikes is between 2 and 100 Hz. Preferably, the interburst frequency between each burst or group of electric pulses or spikes may be in the range of 10 and 100 Hz, in particular in the range of 10 and 60 Hz. For example, the interburst frequency may be 10 Hz, 20 Hz or 40 Hz.

A skilled person will further realize that each burst stimulus comprises at least two spikes or pulses, for example, each burst stimulus can comprise about 2 to about 100 electric pulses, more particularly, about 2 to about 20 electric pulses. Typical for the present invention is that each electric pulse or spike comprises a frequency in the range of 50 Hz to 1000 Hz, in particular in the range of 200 Hz to 1000 Hz, more in particular in the range of 500 to 1000 Hz. In some embodiments, the methods as taught herein thus comprise applying a burst pattern wherein the intraburst frequency within each group of electric pulses is from 50 Hz to 1000 Hz, preferably from 200 Hz to 1000 Hz, more preferably from 500 Hz to 1000 Hz. For example, the intraburst frequency may be 500 Hz.

In some embodiments, the methods as taught herein comprise applying a burst pattern wherein each electric pulse or electric spike within the burst stimulus of the burst pattern has an amplitude from 0.10 mA to 20.0 mA, preferably from 0.10 mA to 15.0 mA, more preferably from 0.10 mA to 13.0 mA, even more preferably from 1.0 mA to 10.0 mA. For example, the amplitude of the electric pulses within the burst stimulus may be 1.0 mA, or 2.0 mA, or 3.0 mA, or 4.0 mA, or 5.0 mA, or 6.0 mA, or 7.0 mA, or 8.0 mA, or 9.0 mA, or 10.0 mA. In preferred embodiments, the amplitude of the electric pulses within the burst stimulus may be within 1.0 mA and 5.0 mA.

In some specific embodiments, the methods as taught herein comprise applying a burst pattern wherein the interburst frequency between each group of electric pulses is 10 Hz and wherein the amplitude of each pulse of each group of electric pulses is 4 mA.

In some embodiments, the methods as taught herein comprise applying a burst pattern wherein each electric pulse or spike within the burst stimulus has a pulse width that ranges from 10 to 5000 μseconds (μs), preferably from 20 to 2000 μs, more preferably from 50 to 1000 μs. For example, a pulse width of 500 μs or of 1000 μs is applied.

In some embodiments, the methods as taught herein comprise applying a burst pattern wherein the interburst frequency between each burst or group of electric pulses or spikes is between 2 and 100 Hz, preferably in the range of 10 and 100 Hz, more preferably in the range of 10 and 60 Hz, wherein the intraburst frequency within each group of electric pulses is from 50 Hz to 1000 Hz, preferably from 200 Hz to 1000 Hz, more preferably from 500 Hz to 1000 Hz, wherein each electric pulse or electric spike within the burst stimulus of the burst pattern has an amplitude from 0.10 mA to 20.0 mA, preferably from 0.10 mA to 15.0 mA, more preferably from 0.10 mA to 13.0 mA, and wherein each electric pulse or spike within the burst stimulus has a pulse width of from 10 to 5000 μseconds (μs), preferably from 20 to 2000 μs, more preferably from 50 to 1000 μs.

In some embodiments, the burst pattern as taught herein has an interburst frequency between each burst or group of electric pulses or spikes from 10 to 60 Hz, an intraburst frequency within each group of electric pulses from 500 Hz to 1000 Hz, an amplitude of each electric pulse or electric spike within the burst stimulus that ranges from 0.10 mA to 13.0 mA, and a pulse width of each electric pulse or spike within the burst stimulus that is from 50 to 1000 μs. For example, the burst pattern as taught herein may have an interburst frequency of 10 Hz, an interburst frequency of 500 Hz, an amplitude of each electric pulse of electric spike with the burst stimulus of 1 mA and a pulse width of each electric pulse or spike within the burst stimulus of 1000 μs. Or, the burst pattern as taught herein may have an interburst frequency of 20 Hz, an interburst frequency of 500 Hz, an amplitude of each electric pulse of electric spike with the burst stimulus of 1 mA and a pulse width of each electric pulse or spike within the burst stimulus of 1000 μs. Or, the burst pattern as taught herein may have an interburst frequency of 40 Hz, an interburst frequency of 500 Hz, an amplitude of each electric pulse of electric spike with the burst stimulus of 1 mA and a pulse width of each electric pulse or spike within the burst stimulus of 1000 μs. Or, the burst pattern as taught herein may have an interburst frequency of 10 Hz, an interburst frequency of 500 Hz, an amplitude of each electric pulse of electric spike with the burst stimulus of 2 mA and a pulse width of each electric pulse or spike within the burst stimulus of 1000 μs. Or, the burst pattern as taught herein may have an interburst frequency of 20 Hz, an interburst frequency of 500 Hz, an amplitude of each electric pulse of electric spike with the burst stimulus of 2 mA and a pulse width of each electric pulse or spike within the burst stimulus of 1000 μs. Or, the burst pattern as taught herein may have an interburst frequency of 40 Hz, an interburst frequency of 500 Hz, an amplitude of each electric pulse of electric spike with the burst stimulus of 2 mA and a pulse width of each electric pulse or spike within the burst stimulus of 1000 μs. Or, the burst pattern as taught herein may have an interburst frequency of 10 Hz, an interburst frequency of 500 Hz, an amplitude of each electric pulse of electric spike with the burst stimulus of 3 mA and a pulse width of each electric pulse or spike within the burst stimulus of 1000 μs. Or, the burst pattern as taught herein may have an interburst frequency of 20 Hz, an interburst frequency of 500 Hz, an amplitude of each electric pulse of electric spike with the burst stimulus of 3 mA and a pulse width of each electric pulse or spike within the burst stimulus of 1000 μs. Or, the burst pattern as taught herein may have an interburst frequency of 40 Hz, an interburst frequency of 500 Hz, an amplitude of each electric pulse of electric spike with the burst stimulus of 3 mA and a pulse width of each electric pulse or spike within the burst stimulus of 1000 μs. Or, the burst pattern as taught herein may have an interburst frequency of 10 Hz, an interburst frequency of 500 Hz, an amplitude of each electric pulse of electric spike with the burst stimulus of 4 mA and a pulse width of each electric pulse or spike within the burst stimulus of 1000 μs. Or, the burst pattern as taught herein may have an interburst frequency of 20 Hz, an interburst frequency of 500 Hz, an amplitude of each electric pulse of electric spike with the burst stimulus of 4 mA and a pulse width of each electric pulse or spike within the burst stimulus of 1000 μs. Or, the burst pattern as taught herein may have an interburst frequency of 40 Hz, an interburst frequency of 500 Hz, an amplitude of each electric pulse of electric spike with the burst stimulus of 4 mA and a pulse width of each electric pulse or spike within the burst stimulus of 1000 μs.

In some embodiments, the methods as taught herein comprise a burst pattern wherein the intraburst frequency is constant during the entire burst pattern. A constant intraburst frequency is to be understood as an intraburst frequency that is within the same range in each burst of the burst pattern. Preferably, a constant intraburst frequency is an intraburst frequency that is the same in each burst of the burst pattern. For example, the intraburst frequency may range for each burst within the burst pattern from 50 Hz to 1000 Hz, preferably from 200 Hz to 1000 Hz, more preferably from 500 Hz to 1000 Hz, wherein the intraburst frequency is the same in each burst of the burst pattern. For example, the intraburst frequency of the burst pattern may be 200 Hz in each burst of the burst pattern, or the intraburst frequency of the burst pattern may be 500 Hz in each burst of the burst pattern. In some embodiments, the intraburst frequency is constant throughout the entire burst pattern and is 500 Hz in each burst of the burst pattern.

In some embodiments, the methods as taught herein comprise applying a burst pattern that comprises a pulse width that is constant for each pulse of each group of electric pulses throughout the entire burst pattern. A constant pulse width is to be understood as a pulse width that is within the same range in each pulse of each group of pulses throughout the burst pattern. Preferably, a constant pulse width is a pulse width that is the same in each pulse of the burst pattern. For example, the pulse width may range for each pulse within the burst pattern from 50 μs to 2000 μs, wherein the pulse width in each pulse is the same. For example, the pulse width of each pulse of the burst pattern may be 1000 μs.

In some embodiments, the methods as taught herein comprise a burst pattern wherein the amplitude of the first electric pulse in a group of electric pulses is smaller than the amplitude of the subsequent electric pulse in said group of electric pulses. In some embodiments, the methods as taught herein comprise a burst pattern wherein the amplitude of the first electric pulse in each group of electric pulses is smaller than the amplitude of the subsequent electric pulse in each group of electric pulses. In some embodiments, the methods as taught herein comprise a burst pattern wherein the amplitude of the first electric pulse of each group of electric pulses is the same, wherein the amplitude of a subsequent electric pulse within the group of electric pulses gradually increases compared to the preceding pulse within said group of electric pulses and wherein a passive charge balance occurs during each interburst interval. Thus, in some embodiments, the amplitude of a subsequent pulse in a group of electrical pulses is higher than the amplitude of its preceding pulse within the same group of electric pulses and the amplitude of each subsequent pulse gradually increases compared to the preceding pulse within said group. In certain embodiments, the amplitude of the electric pulses in a group of electric pulses thus incrementally and gradually increases wherein the first pulse in each group of electric pulses has the lowest amplitude and wherein the amplitude of each subsequent pulse in said group of electric pulses is higher than the amplitude of its preceding pulse. In some embodiments, the gradual increase of the amplitude within a group of electric pulse may be between 0.1 mA and 5 mA, in particular between 0.2 mA and 2 mA, in particular between 0.5 mA and 2 mA, such as for example 0.5 mA, 1.0 mA, 1.5 mA or 2.0 mA. For example, the amplitude of the pulses within a group of electrical pulses may increase from 1 mA to 2 mA, or from 1 mA to 2 mA and further to 3 mA and 4 mA. In some other embodiments, the amplitude of the pulses in a group of electrical pulses may increase from 1 mA for the first pulse to 2 mA for the second pulse, to 3 mA for the third pulse, to 4 mA for the fourth pulse. In some embodiments, a passive recharge balance is present during each interburst interval. As used herein, a “passive recharge balance” refers to a passive restoration of the voltage difference that occurs after applying monophasic pulses which result in a voltage difference over the cell membrane. The voltage difference over the cell membrane is then passively restored by exchange of electrolytes in the cell.

In some embodiments, the methods as taught herein comprise a burst pattern wherein the amplitude of each electric pulse in a group of electric pulses is the same. Preferably, the amplitude of each electric pulse in the whole burst pattern is the same, followed by an active recharge burst stimulation during each interburst interval. As used herein, an “active recharge balance” is to be understood as the active restoration of the voltage difference after applying biphasic, i.e. individually charge balanced, pulses, wherein the positive peak and the negative peak balance each other out, thereby actively restoring the voltage difference.

In some embodiments, the methods as taught herein comprise a burse pattern wherein each group of electric pulses or each burst comprises at least two electric pulses or spikes. In some embodiments, each group of electric pulses or burst comprises at least three, preferably at least four electric pulses or spikes. In some embodiments, each group of electric pulses or burst may comprise from 2 to 100 electric pulses or spikes, preferably from 2 to 10 electric pulses or spikes. In some embodiments, the number of pulses in each group of electric pulses or in each burst is the same in the whole burst pattern. For example, the burst pattern as applied in the methods as taught herein may comprise 2 spikes in each burst, or it may comprise 3 spikes in each burst, or it may comprise 4 spikes in each burst.

In some embodiments, the methods as taught herein comprise determining the nature of the dysfunction of the autonomous nervous system in the subject and adjusting one or more of the parameters of methods as taught herein, in particular adjusting one or more of the application location, the amplitude and the interburst frequency of the burst pattern to specifically correct the dysfunction in the subject. For example, the location of the application of the burst pattern can be adjusted by adjusting the location of the one or more electrodes of the one or more electrical leads that are operably linked to the pulse generator. Or, the burst pattern that is applied to the subject can be adjusted by adjusting the amplitude of the electrical pulses of the burst that are applied, or by adjusting the interburst frequency.

In certain embodiments, the methods as taught herein further comprise detecting or determining one or more of the bladder pressure (pᵥₑₛ), proximal urethral pressure (pᵤᵣₐ₁) and mid urethral pressure (pᵤᵣₐ₂) of the subject. In some embodiments, the methods as taught herein thus further comprise detecting or determining the bladder pressure (pᵥₑₛ) of the subject. In some embodiments, the methods as taught herein thus further comprise detecting or determining proximal urethral pressure (pᵤᵣₐ₁) of the subject. In some embodiments, the methods as taught herein thus further comprise detecting or determining mid urethral pressure (pᵤᵣₐ₂) of the subject. In some further embodiments, the bladder pressure (pᵥₑₛ), the proximal urethral pressure (pᵤᵣₐ₁) and the mid urethral pressure (pᵤᵣₐ₂) of the subject may be determined. Difference in one of these pressures before and after sacral neuromodulation can be used to better determine whether continence or full voiding in a patient is achieved after applying the method as taught herein.

A patient's vesicle (bladder) and urethral pressures can be measured by introducing a catheter into the urethra and positioning pressure sensors of the catheter at the desired measurement sites in the urethra and/or bladder.

The urethral pressure can be measured in the proximal end of the urethra (proximal urethral pressure; pᵤᵣₐ₁) or in the mid part of the urethra (pᵤᵣₐ₂).

As corroborated by the examples, the inventors here found that by applying the burst pattern to the subject as disclosed in the methods as taught herein, the bladder pressure (pᵥₑₛ), the proximal urethral pressure (pᵤᵣₐ₁), the mid urethral pressure (pᵤᵣₐ₂), or a combination thereof can be increased in the subject, resulting in a significant improvement of the disease symptoms.

It is apparent that there have been provided in accordance with the invention products, methods, and uses, that provide for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples. Also other characteristics and advantages of the invention will appear in the following examples and the figures illustrating the invention.

### EXAMPLES

Methods

Study Group

This monocentric prospective pilot study was approved by the local medical ethics research committee (14/50/526). Between March 2021 and July 2021 six patients suffering from urinary tract dysfunction underwent a sacral neuromodulation (SNM) implantation at the Antwerp University Hospital. All patients provided written informed consent with guarantees of confidentiality.

Protocol

The unilateral lead (model 3889) was placed close (approximately within 5 mm) to the sacral spinal nerve according to the standardized tined lead placement technique using the curved stylet under general anaesthesia (Matzel et al., Neuromodulation 2017, 20(8): 816-24). Patients were tested with an external pulse generator for three weeks with continuous subsensory conventional stimulation parameters (210 μs, 14 Hz) during which improvement in key voiding diary variables was examined: for chronic non-obstructive urinary retention (NOUR) the post void residual was determined by catheterization and for overactive bladder (OAB) the number of incontinence episodes was registered. Patients achieving >50% improvement received an Implantable Pulse Generator (IPG).

After emptying the patient's bladder, it was filled with normal saline (0.9% NaCl) to 50% of their maximum cystometric capacity (MCC), as previously determined during urodynamic study. Data were collected using a 7-French diagnostic electrode unisensor catheter (UniTip, Laborie Medical®, Enschede, The Netherlands) introduced transurethrally. This custom-made catheter (UniTip) has radio opaque markers with a microtip electrode in the bladder, 3 microtip electrodes—with an inter-electrode space of 2 mm—in the urethra, and a filling lumen (which was closed after the bladder was filled). The catheter was connected to the ambulatory urodynamic unit (Laborie Medical®, Enschede, The Netherlands). Bladder pressure (pᵥₑₛ), proximal urethral pressure (pᵤᵣₐ₁) and mid urethral pressure (pᵤᵣₐ₂) were measured.

During this pilot study all patients received four SNM stimulation paradigms under general anaesthesia. Stimulation paradigms were applied in a random order using randomization software. Con-SNM was performed incrementally using the Verify device (Medtronic®, Fridley, MN, USA): 1 to 5 mA, 210 μs and 14 Hz. Burst stimulation was administered using the St. Jude Medical™ 8 channel adapter (Abbott Laboratories®, Chicago, Illinois, USA). In the burst stimulation protocols, incremental stimulation intensities were applied (1-2-3-4 mA) with different interburst frequencies (10-20-40 Hz). The intraburst frequency (500 Hz) and pulse width (1000 μsec) remained constant. Between different stimulation paradigms a rest period of 20 seconds was inserted to make sure pressures returned to baseline again. For each stimulation protocol the differences in bladder pressure (Δpᵥₑₛ), proximal and mid urethral pressure (resp. Δpᵤᵣₐ₁, and Δpᵤᵣₐ₂) were calculated.

Statistical Analysis

Statistical analysis was performed with JMP Pro 15 (SAS Institute®, Cary, North Carolina, US). The mean differences in proximal and mid urethral pressure (resp. Δpᵤᵣₐ₁ and Δpᵤᵣₐ₂) and the differences in bladder pressure (Δpᵥₑₛ) between the incrementally increasing stimulation amplitudes were analysed for the different stimulation intensities and paradigms by a mixed model analysis (MMA). Δpᵤᵣₐ₁, Δpᵤᵣₐ₂ and Δpᵥₑₛ were separately modelled as outcome, and stimulation intensity and paradigm were modelled as fixed effects with a random intercept for each patient. Based upon the Wald test (p>0.05) no random slope was needed. The interaction term of the fixed effects (e.g., regression coefficient or slope) was kept if significant. Due to non-normal data distribution a log transformation was performed for Δpᵤᵣₐ₁, Δpᵤᵣₐ₂ and Δpᵥₑₛ. If significant, a Tukey HSD all pairwise comparisons post hoc analysis was performed. Statistically significant results were defined as p<0.0083 (i.e. p<0.05/6) as adjusted significance tests for pairwise comparisons.

Secondly, a comparison of Burst paradigms with a uniform total charge was performed. Total charge was defined as the charge per pulse multiplied by the number of pulses per second. This comparison with a total charge of 200 μC/s charge per second was performed on: A) 10 Hz interburst frequency with a 4 mA stimulation amplitude; B) 20 Hz interburst frequency with a 2 mA stimulation amplitude; and C) 40 Hz interburst frequency with a 1 mA stimulation amplitude. One-way Repeated Measures ANOVA was performed to compare the effect of the stimulation paradigm (independent variable) on the different pressure measurements (dependent variable). Mauchly's Test of Sphericity showed no violation of sphericity. A p-value <0.05 was considered statistically significant. Partial Eta Squared was examined to investigate to which extent the variability in pressure changes was accounted for by the different Burst paradigms.

Results

Six patients who underwent a SNM implant were included in the study, of which five patients suffered from OAB and one patient suffered from NOUR with minor detrusor contractions and ability to void. All patients were female, and the mean age was 65 (±15.6; 42-80) years old. Patient characteristics are shown in Table 1.

TABLE 1
Patients characteristics of the patients undergoing SNM.

| | | Age | Sacral | Unilateral |
|---|---|---|---|---|
| | Disorder | (year) | nerve | side |
| Patient 1 | OABwet | 53 | S3 | left |
| Patient 2 | OABwet | 42 | S3 | left |
| Patient 3 | NOUR | 80 | S3 | right |
| Patient 4 | OABwet | 75 | S3 | right |
| Patient 5 | OABwet | 79 | S3 | right |
| Patient 6 | OABwet | 61 | S3 | right |

OABwet = overactive bladder with urinary incontinence;
NOUR = non-obstructive urinary retention

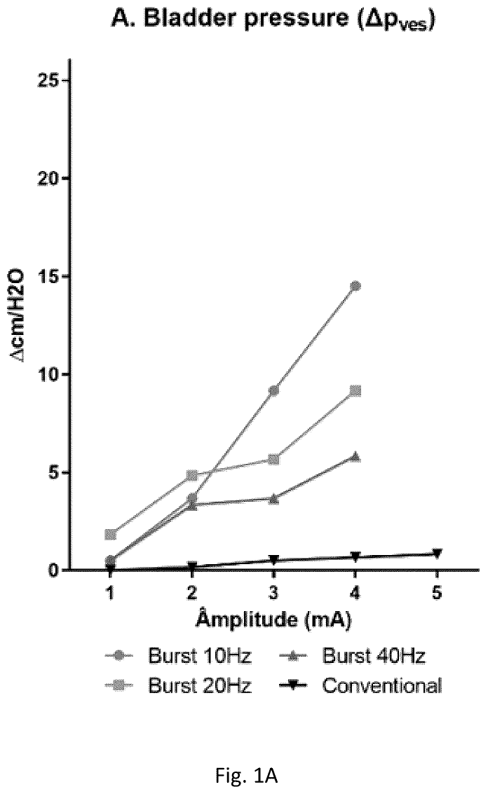

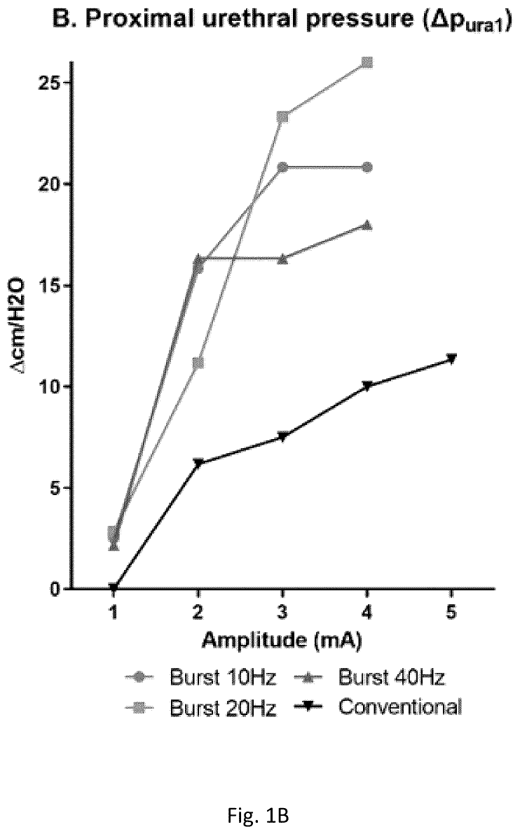

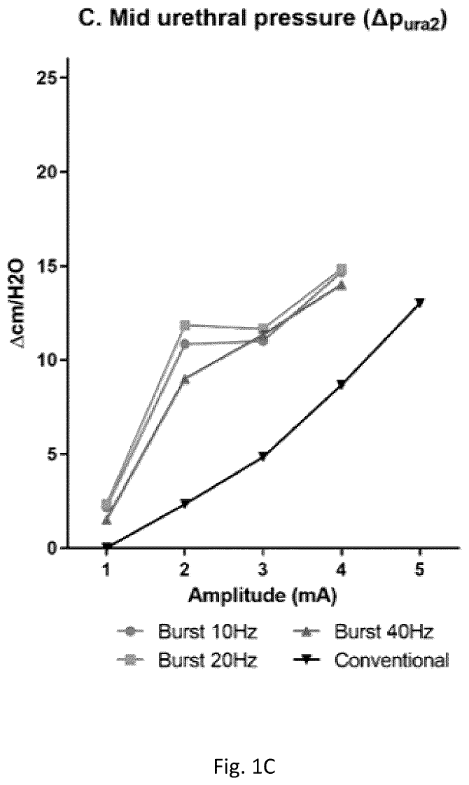

Data of the cystometry are shown in FIG. 1. All four stimulation paradigms showed an increase in bladder pressure as the stimulation amplitude increased (p<0.0001), however the increase in bladder pressure over the increased stimulation intensities (e.g., regression coefficient or slope) varied between stimulation paradigms (FIG. 1A; interaction effect p=0.0078). Using a Tukey post hoc analysis, all Burst paradigms were significantly different as compared to Con-SNM (interburst 10 Hz p<0.0001; interburst 20 Hz p<0.0001; interburst 40 Hz p=0.0003), indicating that Burst paradigms showed a higher increase in bladder pressure per increase in stimulation intensity compared to the conventional stimulation. Interestingly, bladder pressure rise was inversely correlated to SNM interburst frequency at higher amplitudes (3 and 4 mA).

In addition, all paradigms showed an increase in urethral pressure as stimulation amplitude increased (FIG. 1B-C; Δpᵤᵣₐ₁ p<0.0001; Δpᵤᵣₐ₂ p<0.0001). The urethral pressure was significantly different between the different paradigms (Δpᵤᵣₐ₁ p<0.0001; Δpᵤᵣₐ₂ p=0.0007). Using Tukey post hoc analysis for proximal urethral pressure differences, all Burst paradigms were significantly higher as compared to Con-SNM (interburst 10 Hz p<0.0001; interburst 20 Hz p<0.0001; interburst 40 Hz p=0.0070). For mid urethral pressure, Burst paradigms with an interburst frequency of 10 Hz and 20 Hz were significantly different from Con-SNM (resp. p=0.0014 and p=0.0036), where an interburst frequency of 40 Hz was not (p=0.1724). No difference in regression coefficient was found between stimulation paradigms (proximal urethral pressure p=0.6642; mid urethral pressure p=0.8052), indicating no statistically significant difference in regression coefficient. Interestingly, the pressure rise for Burst paradigms in the proximal part of the urethra is observed to be higher as compared to pressure rise in the middle part over the urethra, whereas pressure rise for Con-SNM seemed nearly comparable in the proximal (FIG. 1B) and mid (FIG. 1C) urethra.

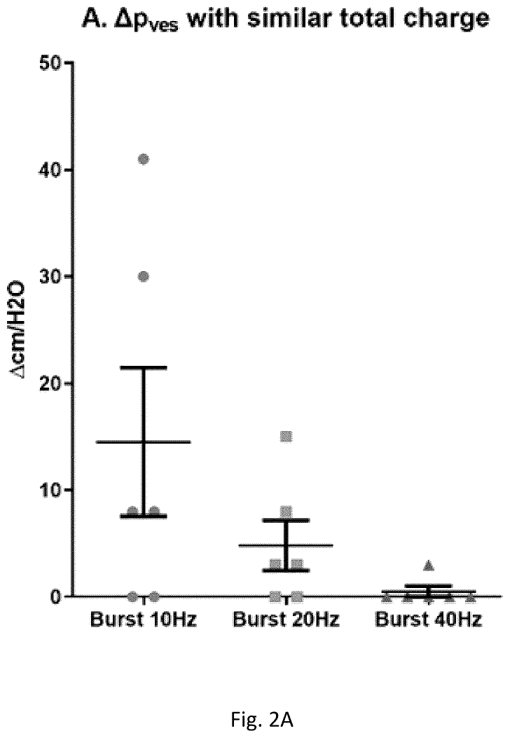

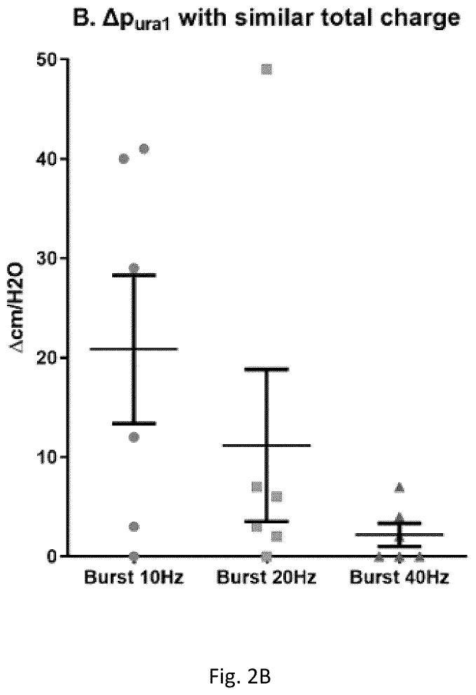

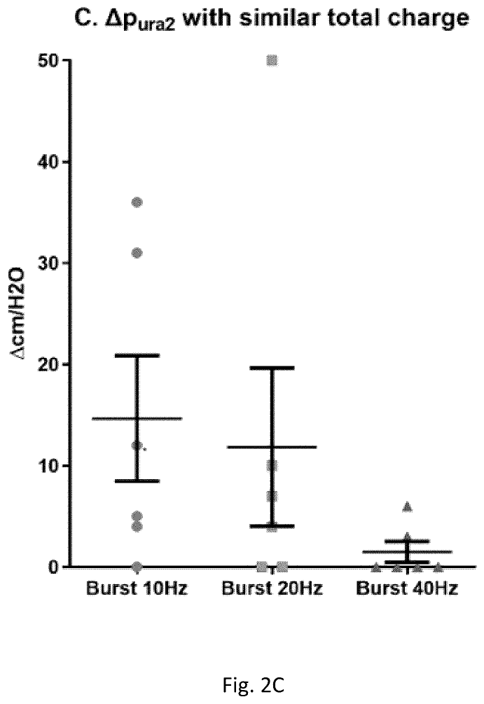

FIG. 2 shows the Burst paradigms with similar total charge. For both bladder pressure and urethral pressure, Burst stimulation at 10 Hz with a 4 mA amplitude evoked the highest rise in pressure and 40 Hz with a 1 mA amplitude evoked the lowest rise in pressure, albeit not significant due to the low power (Δpᵥₑₛ p=0.055; Δpᵤᵣₐ₁ p=0.100; Δpᵤᵣₐ₂ p=0.143). However, effect sizes showed a relatively big practical effect which assumes a tendency towards significance with a higher power (Δpᵥₑₛ pη²=0.439; Δpᵤᵣₐ₁ pη²=0.368; Δpᵤᵣₐ₂ pη²=0.322).

1. A method of ensuring pelvic health and/or treating a disease or disorder characterized by a dysfunctional autonomic nervous system in a human subject with neuromodulation by applying electric pulses to the subject through a set of one or more electrodes of an electrical lead that is operably linked to a pulse generator,
wherein said method comprises applying a burst pattern comprising a plurality of groups of electric pulses to the subject through a set of one or more electrodes of an electrical lead located in the proximity of the sacral plexus and/or pelvic plexus of the human subject.

2. The method according to claim 1, wherein the dysfunctional autonomic nervous system is selected from the group consisting of a muscle control disorder of the colon or bladder, a sexual dysfunction, an inflammatory disorder of the bladder or intestines, chronic visceral pain, chronic bladder pain and chronic pelvic pain.

3. The method according to claim 2, wherein the muscle control disorder of the colon or bladder is a muscle disorder of one or more sphincters of the colon or bladder.

4. The method according to claim 1, comprising a step of positioning the one or more electrodes of the electrical lead in the proximity of the sacral and/or pelvic plexus of the subject by guiding the one or more electrodes through the third or fourth sacral foramen.

5. The method according to claim 1, wherein the interburst frequency between each group of electric pulses is between 10 and 60 Hz, wherein the intraburst frequency within each group of electric pulses is from 500 to 1000 Hz, and wherein each electric pulse of the burst pattern has an amplitude of from 0.10 to 13.0 mA and a pulse width of from 50 to 1000 μseconds.

6. The method according to claim 5, wherein the intraburst frequency is constant throughout the entire burst pattern.

7. The method according to claim 6, wherein the intraburst frequency is 500 Hz.

8. The method according to claim 5, wherein the amplitude of the first pulse of each group of electric pulses is the same, wherein the amplitude of a subsequent pulse within a group of electric pulses gradually increases compared to the preceding pulse within said group and wherein a passive charge balance occurs during each interburst interval.

9. The method according to claim 5, wherein the amplitude of each pulse in the burst pattern is the same.

10. The method according to claim 5, wherein the pulse width of each pulse of each group of electric pulses is constant throughout the entire burst pattern.

11. The method according to claim 5, wherein the pulse width of each pulse of each group of electric pulses is 1000 μseconds.

12. The method according to claim 5, wherein the interburst frequency between each group of electric pulses is 10 Hz and the amplitude of each pulse of each group of electric pulses is 4 mA.

13. The method according to claim 5, wherein the pulse generator is an implantable pulse generator.

14. The method according to claim 1, wherein the method further comprises detecting or determining one or more of the bladder pressure (pᵥₑₛ), proximal urethral pressure (pᵤᵣₐ₁) and mid urethral pressure (pᵤᵣₐ₂) of said subject.

15. The method according to claim 1, wherein applying the burst pattern to the subject increases the bladder pressure, proximal urethral pressure, the mid urethral pressure, or a combination thereof.

16. The method according to claim 1 which comprises determining the nature of the dysfunction of the of said autonomic nervous system in said patient and adjusting one or more of the application location, amplitude and interburst frequency of said burst pattern to specifically correct said dysfunction.
